(19) United States
(12) Patent Application Publication
(10) Pub. No.: US 2010/0094074 A1
(43) Pub. Date: Apr. 15, 2010

(54) Brachytherapy apparatus and methods employing expandable medical devices comprising fixation elements

(75) Inventors: Joseph L. Mark, Indianapolis, IN (US); Zachary R. Nicoson, Indianapolis, IN (US)

Correspondence Address: BINGHAM MCCUTCHEN LLP, 2020 K Street, N.W., Intellectual Property Department, WASHINGTON, DC, 20006, US

(73) Assignee: Hologic Inc.

(21) Appl. No.: 12/285,661

(22) Filed: Oct. 10, 2008

(51) Int. Cl. A61M 36/04

(52) U.S. Cl. 600/3

(57) ABSTRACT

Brachytherapy apparatus and methods for performing brachytherapy employing expandable members with at least one external fixation element, offering precise therapy due to rotational and/or longitudinal stability of the expandable member.

### BACKGROUND OF THE INVENTION

This invention relates generally to brachytherapy apparatus and methods employing expandable medical devices comprising at least one fixation element.

Treatment of Medical Disorders Using Expandable Medical Devices

Medical balloons are one type of expandable medical device that are widely-used in a number of medical procedures. Typically, an uninflated medical balloon is inserted into a space within the patient's body. When the medical balloon is inflated, the volume of the medical balloon expands, and the space is similarly expanded. In procedures such as angioplasty, the medical balloon may be used to open a collapsed or blocked artery.

Medical balloons are often employed with catheters, with or without stents, to treat strictures, stenoses, and/or narrowings in various parts of the human body. Devices with varying designs have been utilized for angioplasty, including stents and grafts or combination stent/grafts.

Procedures involving balloon catheters include percutaneous transluminal angioplasty (“PTA”) and percutaneous transluminal coronary angioplasty (“PTCA”), which may be used to reduce arterial build-up, such as that caused by the accumulation of atherosclerotic plaque. In those procedures, a balloon catheter is typically passed over a guidewire to a stenosis with the aid of a guide catheter. The guidewire extends from a remote incision to the site of the stenosis, and typically across the lesion. The balloon catheter is passed over the guidewire, and ultimately positioned across the lesion.

Once the balloon catheter is positioned appropriately across the lesion, often with fluoroscopic guidance, the balloon is inflated. As a result, the plaque of the stenosis is broken and the arterial cross section is increased. The balloon is then deflated and withdrawn over the guidewire into the guide catheter, and removed from the patient's body of the patient.

Treatment of Proliferative Disorders

Treatment of proliferative disorders (disorders including or characterized by rapid or abnormal cell growth or proliferation, including tumors, restenosis, abnormal angiogenesis, hyperplasia, and the like) has become increasingly sophisticated in recent years, and improvements in surgical, chemotherapeutic, and brachytherapeutic techniques have led to better outcomes in patients suffering from such disorders. Malignant tumors are often treated by removing as much of the tumor as possible with surgical resection. Yet, the therapeutic value of this procedure is reduced if tumor cells infiltrate into normal tissue surrounding the tumor. To combat this, surgical resection is often supplemented with radiation therapy whereby the residual tumor margin is targeted after resection.

The supplemental radiation therapy is administered through any number of methods, ranging from external beam radiation, stereotactic radiosurgery, and permanent or temporary brachytherapy. “Brachytherapy” refers to radiation therapy delivered by a spatially-confined source of therapeutic rays inserted into a mammalian body at or near a tumor or other proliferative tissue disease site. Due to the proximity of the radiation source, brachytherapy offers the advantage of delivering a more localized dose to the target tissue region. For example, brachytherapy can be performed by implanting radiation sources directly into the tissue to be treated. Brachytherapy is most appropriate where: (1) malignant tumor regrowth occurs locally, within 2 or 3 cm of the original boundary of the primary tumor site; (2) radiation therapy is a proven treatment for controlling the growth of the malignant tumor; and (3) there is a radiation dose-response relationship for the malignant tumor, but the dose that can be given safely with conventional external beam radiotherapy is limited by the tolerance or normal tissue. In brachytherapy, radiation doses are highest in close proximity to the radiotherapeutic source, providing a high tumor dose while sparing surrounding normal tissue. Brachytherapy is useful for treating malignant brain and breast tumors, among others.

Interstitial brachytherapy is often carried out using radioactive seeds, such as ¹²⁵I seeds. Unfortunately, these seeds produce variable dose distributions. To achieve a minimum prescribed dosage throughout a target region of tissue, high activity seeds are often used. This often results in very high radiation doses being delivered to regions closest to the seed(s). That, in turn, often leads to radionecrosis in healthy tissue.

Prior art brachytherapy devices have provided a number of advancements in the delivery of radiation to target tissue. For example, Williams U.S. Pat. No. 5,429,582 (“Williams”), incorporated herein in its entirety for all purposes, describes a method and apparatus for treating tissue surrounding a surgically-excised tumor with radioactive emissions to kill any cancer cells that may be present in the tissue surrounding the excised tumor. To deliver the radioactive emissions, Williams provides a catheter having an inflatable balloon, such as those discussed above, at its distal end that defines a distensible reservoir. After the tumor is surgically removed, the surgeon introduces the balloon catheter into the surgically-created pocket where the tumor had resided. The balloon is then inflated by injecting a fluid having one or more radionuclides into the distensible reservoir via a lumen in the catheter.

The apparatus described in Williams solved some of the problems found when using radioactive seeds for interstitial brachytherapy, but left some problems unresolved. The absorbed dose rate at a target point exterior to a radioactive source is inversely proportional to the square of the distance between the radiation source and the target point. As a result, where the radioactive source has sufficient activity to deliver a prescribed dose, e.g., two centimeters into the target tissue, the tissue directly adjacent the wall of the distensible reservoir, where the distance to the radioactive source is very small, may still be overly “hot” to the point where healthy tissue necrosis may result. Generally, the amount of radiation desired by the physician is a certain minimum amount that is delivered to a region up to about two centimeters away from the wall of the excised tumor. It is desirable to keep the radiation that is delivered to the tissue in the target treatment region within a narrow absorbed dose range to prevent over-exposure to tissue at or near the reservoir wall, while still delivering the minimum prescribed dose at the maximum prescribed distance from the reservoir wall.

U.S. Pat. No. 6,413,204 to Winkler et al., incorporated herein in its entirety for all purposes, provides an apparatus that delivers radiation from a radioactive source to target tissue within the human body with a desired intensity and at a predetermined distance from the radiation source, without over-exposure of body tissues disposed between the radiation source and the target. The apparatus includes a catheter body member having a proximal end and distal end, an inner spatial volume disposed proximate to the distal end of the catheter body member, an outer spatial volume defined by an expandable surface element, such as a balloon, disposed proximate to the distal end of the body member in a surrounding relation to the inner spatial volume, and a radiation source disposed in the inner spatial volume. The inner and outer spatial volumes are configured to provide an absorbed dose within a predetermined range throughout a target tissue. The target tissue is located between the outer spatial volume expandable surface and a minimum distance outward from the outer spatial volume expandable surface. The predetermined dose range is defined as being between a minimum prescribed absorbed dose for delivering therapeutic effects to tissue that may include cancer cells, and a maximum prescribed absorbed dose above which healthy tissue necrosis may result.

In years past, brachytherapy often calculated the desired radiation dose based on the characteristics of the brachytherapy applicator (device), the radiation source, and the surrounding tissue. Yet, the actual dose delivered was not tested to assure that over- and/or under-treatment did not occur. For example, if the radiation source is a radioactive seed positioned in the center of an expanded balloon, the calculated dose is based on the central positioning of the radiation source. If for some reason the radioactive seed was positioned off center, prior art brachytherapy devices had no means to determine that this harmful situation was occurring. Prior art brachytherapy devices also lacked the ability to directly sense the surrounding tissue and determine the effectiveness of the proliferative tissue disorder treatment. The implantable radiotherapy/brachytherapy radiation-detecting apparatus and methods described in U.S. Pat. No. 7,354,391 to Stubbs, incorporated herein in its entirety for all purposes, remedied that situation by offering a means to deliver and monitor radioactive emissions applied within a mammalian body. There, the device employed included a catheter body member having a proximal end, a distal end, and an outer spatial volume disposed proximate to the distal end of the body member. A radiation source was preferably positioned in the outer spatial volume, and a treatment feedback sensor was disposed on the device.

U.S. Pat. No. 6,482,142 to Winkler et al. (“the '142 Patent”), incorporated herein in its entirety for all purposes, provides brachytherapy apparatus for delivering radioactive emissions in an asymmetric fashion to target tissue surrounding a surgical extraction site. The apparatus includes an expandable outer surface element defining an apparatus spatial volume, a radiation source disposed within the apparatus volume, and a means for providing predetermined asymmetric isodose profile within the target tissue. The brachytherapy apparatus of the '142 Patent include an expandable outer surface defining a three-dimensional apparatus volume configured to fill an interstitial void created by the surgical extraction of diseased tissue and define an inner boundary of the target tissue being treated and a radiation source disposed completely within the expandable outer surface and located so as to be spaced apart from the apparatus volume, the radiation source further being asymmetrically located and arranged within the expandable surface to provide predetermined asymmetric isodose curves with respect to the apparatus volume. The brachytherapy apparatus of the '142 Patent may include an asymmetric radiation shield spaced apart from the radiation source that provides predetermined asymmetric isodose curves with respect to the apparatus volume.

The '142 Patent also provides surgical apparatus for providing radiation treatment to target tissue including an expandable outer surface defining an apparatus volume and a radiation source replaceably disposable within the expandable outer surface, the radiation source comprising a plurality of solid radiation sources arranged to provide predetermined asymmetric isodose curves within the target tissue. The plurality of solid radiation sources may be spaced apart on a single elongate member shaped to provide asymmetric placement of the spaced apart solid radiation sources with respect to a longitudinal axis through the apparatus volume, or may be provided on at least two elongate members extending into the apparatus volume, at least one of the elongate members being shaped to provide asymmetric placement of a radiation source with respect to a longitudinal axis through the apparatus volume. In the surgical apparatus, at least one of the plurality of solid radiation sources may have a different specific activity from at least one other solid radiation source.

U.S. Pat. No. 5,913,813 to Williams et al. (“the '813 Patent”), incorporated herein in its entirety for all purposes, discloses apparatus for delivering radioactive emissions to a body location within a uniform radiation profile, by delivering a desired radiation dose at a predetermined radial distance from a source of radioactivity by providing a first spatial volume at the distal end of a catheter and a second spatial volume defined by a surrounding of the first spatial volume by a polymeric film wall where the distance from the spatial volume and the wall is maintained substantially constant over their entire surfaces. In those apparatus, one of the inner and outer volumes is filled with either a fluid or a solid containing a radionuclide(s) while the other of the two volumes is made to contain either a low radiation absorbing material, e.g., air or even a more absorptive material, such as an x-ray contrast fluid. Where the radioactive material comprises the core, the surrounding radiation absorbing material serves to control the radial profile of the radioactive emissions from the particular one of the inner and outer volumes containing the radionuclide(s) so as to provide a more radially uniform radiation dosage in a predetermined volume surrounding the outer chamber. Where the core contains the absorbent material, the radial depth of penetration of the radiation can be tailored by controlling the core size.

While expandable members, such as medical balloons, continue to offer great advantages in treating a number of human ailments, such as those exemplified above, employment of the expandable members brings with it certain disadvantages. Included within the disadvantages is a lack of stability, including rotational stability and longitudinal stability. For example, when conventional expandable members, such as balloons, are deployed, the member may rotate or spin away, over time, from its initial placement location. Even if the conventional expandable member does not rotate or spin, it may float or slip away from its initial placement location. This is detrimental to the therapy because the target area may not receive consistent therapy. With regard to brachytherapy, this is especially detrimental because the therapeutic radiation may not be consistently administered to the target tissue and/or because the radiation may move away from the target tissue to healthy tissue that can because damaged due to the unwarranted radiation.

Accordingly, there is a need for brachytherapy apparatus and methods employing an expandable member with increased rotational and/or longitudinal stability.

### SUMMARY OF THE INVENTION

The present invention provides expandable medical devices that are characterized by increased rotational and/or longitudinal stability during use in the treatment of medical disorders, brachytherapy apparatus employing such devices, and methods for performing brachytherapy employing such devices.

In one embodiment, the device is an expandable member for placement within a patient that includes an outer surface and at least one external fixation element affixed on the outer surface, wherein the external fixation element contacts tissue within the patient to provide rotational and/or longitudinal stability of the expandable member within the patient. The external fixation element may take any configuration that achieves the improved rotational and/or longitudinal stability.

In one embodiment, the device is an expandable member for placement within a patient that includes an outer surface and at least one external fixation element on said outer surface, wherein upon expansion of the expandable member, the external fixation element projects from the outer surface to contact tissue within a patient, thereby providing rotational and/or longitudinal stability of the expandable member within the patient. The external fixation element may take any configuration that achieves the improved rotational and/or longitudinal stability. The external fixation element, prior to expansion, may be situated in any manner relative to the expandable member, including being recessed within the expandable member and/or nesting proximal to the expandable member.

In another embodiment, the present invention includes a brachytherapy apparatus for delivering radioactive emissions to a patient, including an expandable member for placement within a patient including an outer surface and at least one external fixation element affixed to the outer surface, a catheter including a proximal end, a distal end, and spatial volume at the distal end, wherein the spatial volume is defined by the expandable member, and a radiation source position disposed in the spatial volume, wherein the external fixation element contacts tissue within the patient to provide stability of the expandable member within the patient. The external fixation element may take any configuration that achieves the improved rotational and/or longitudinal stability.

In another embodiment, the present invention includes a brachytherapy apparatus for delivering radioactive emissions to a patient, including an expandable member for placement within a patient including an outer surface and at least one external fixation element, wherein upon expansion of the expandable member the external fixation element projects from the outer surface to contact tissue within the patient, a catheter including a proximal end, a distal end, and spatial volume at the distal end, wherein the spatial volume is defined by the expandable member, and a radiation source position disposed in the spatial volume, wherein the external fixation element contacts tissue within the patient to provide stability of the expandable member within the patient. The external fixation element, prior to expansion, may be situated in any manner relative to the expandable member, including being recessed within the expandable member and/or nesting proximal to the expandable member. The external fixation element may take any configuration that achieves the improved rotational and/or longitudinal stability.

In yet another embodiment, the present invention includes a method for performing a brachytherapy procedure in a patient, including inserting into the patient a catheter including a proximal end, a distal end, and spatial volume at the distal end, wherein the spatial volume is defined by an expandable member including an outer surface and at least one external fixation element on the outer surface; inflating or expanding the expandable member to a volume sufficient to cause the external fixation element(s) to contact tissue within the patient; inserting a radiation source in the spatial volume of the catheter; and removing the radiation source and the catheter from the patient, wherein the contact is sufficient to hinder movement of the expandable member within the tissue. The external fixation element may be affixed to the outer surface of the expandable member and/or may project from the outer surface upon expansion. The external fixation element may take any configuration that achieves the improved rotational and/or longitudinal stability. The external fixation element, prior to expansion, may be situated in any manner relative to the expandable member, including being recessed within the expandable member and/or nesting proximal to the expandable member.

### BRIEF DESCRIPTION OF THE DRAWINGS

The invention will be more fully understood from the following detailed description taken in conjunction with the accompanying drawings:

FIG. 1 illustrates a lack of rotational stability often encountered with conventional expandable members. FIG. 1A illustrates the positioning of the conventional expandable member immediately after placement. FIG. 1B illustrates the positioning of the conventional expandable member following placement and onset of rotation.

FIG. 2 illustrates a lack of longitudinal stability often encountered with conventional expandable members. FIG. 2A illustrates the positioning of the conventional expandable member immediately after placement. FIG. 2B illustrates the positioning of the conventional expandable member following placement and onset of longitudinal movement.

FIG. 3 illustrates a lack of rotational and longitudinal stability often encountered with conventional expandable members. FIG. 3A illustrates the positioning of the conventional expandable member immediately after placement. FIG. 3B illustrates the positioning of the conventional expandable member following placement and onset of rotation and longitudinal movement.

FIG. 4 illustrates an embodiment of the invention. FIG. 4A illustrates an embodiment of the invention wherein an expandable member with eight external fixation elements (spikes) recessed within the member is depicted prior to expansion. FIG. 4B illustrates an embodiment of the invention wherein an expandable member with eight external fixation elements (spikes) is depicted following expansion.

FIG. 5 illustrates an embodiment of the invention. FIG. 5A illustrates an embodiment of the invention wherein an expandable member with two external fixation elements (spikes) nesting proximal to the member is depicted prior to expansion. FIG. 5B illustrates an embodiment of the invention wherein an expandable member with two external fixation elements (spikes) is depicted following expansion.

FIG. 6 illustrates an embodiment of the invention wherein an expandable member with two external fixation elements (wings) is depicted from several views. FIG. 6A illustrates an embodiment of the invention wherein the expandable member and its fixation elements (wings) are viewed from the side. FIG. 6B illustrates an embodiment of the invention wherein the expandable member and its fixation elements (wings) are viewed from the top. FIG. 6C illustrates an embodiment of the invention wherein the expandable member and its fixation elements (wings) are viewed from an end.

FIG. 7 illustrates an embodiment of the invention wherein an expandable member with two external fixation elements are affixed on the outer surface of the expandable member.

FIG. 8 illustrates a conventional brachtherapy apparatus wherein a radiation emitting source is contained within a spatial volume.

FIG. 9 illustrates a brachytherapy apparatus of the present invention for delivering radioactive emissions to mammalian tissue wherein a radiation emitting source is contained within a spatial volume. FIG. 9A illustrates an embodiment of the invention wherein the brachytherapy device has an expandable member with eight external fixation elements (spikes) recessed within the member, depicted prior to expansion. FIG. 9B illustrates an embodiment of the invention wherein the brachytherapy device has an expandable member with eight external fixation elements (spikes), depicted following expansion.

### DETAILED DESCRIPTION OF THE PREFERRED EMBODIMENTS

The present invention provides brachytherapy apparatus and methods employing stable expandable members comprising at least one fixation element. The expandable members of the present invention achieve greater rotational and/or longitudinal stability over convention expandable members due to the fixation element(s).

As used herein, the term “expandable member” includes any device that may be expanded, such as a medical balloon. It will be understood that the term “balloon” is intended to include distensible devices which can be, but need not be, constructed of elastic material. Exemplary balloons include the variety of distensible devices designed for use with surgical catheters. In use, expansion of the expandable member may occur by any means, including air expansion and/or liquid expansion. The expandable member may be fluid-permeable, fluid-impermeable, and/or fluid-semi-permeable, depending on the needs of the treatment.

For example, an expandable member may be constructed of a solid material that is substantially impermeable to active components of a treatment fluid (e.g., radiation source material) with which it can be filled, and is also impermeable to body fluids (e.g., blood, cerebrospinal fluid). An impermeable expandable member is useful in conjunction with a radioactive treatment fluid to prevent the radioactive material from escaping the treatment device and contaminating the therapeutic site or tissues of the patient.

Alternatively, an expandable member may be constructed such that it is permeable to a treatment agent, permitting a treatment agent to pass out of the member and into, for example, a body lumen, body cavity, or therapeutic site. Permeable expandable members are useful when the treatment agent is a drug, such as a chemotherapeutic drug which must contact tissue to be effective.

Treatment agents may also be delivered from the surface of an expandable member to the surrounding tissue.

Generally, it is preferable that the expandable member has a shape that permits the member to conform to the body cavity or site in which it is to be expanded. For example, a generally spherical cavity can be filled with a substantially spherical member, whereas an elongated member is suitable for an elongated cavity, such as a blood vessel. Irregular member shapes may also be appropriate, depending on the needs of the therapy.

In certain embodiments, the expandable member is selected such that upon expansion the member does not compress the tissue which is being treated nor the surrounding tissue. For example, in one embodiment, when the expandable member is placed within a cavity left by surgical removal of tissue, the member is not expanded to assize substantially larger than the size of the cavity. However, in certain other embodiments, the expandable member is expanded so as to compress tissue. For example, when the proliferative disorder being treated is restenosis of a blood vessel, the member is expanded to a size large enough to compress the excess tissue, and may also provide chemotherapy, brachytherapy, or the like..

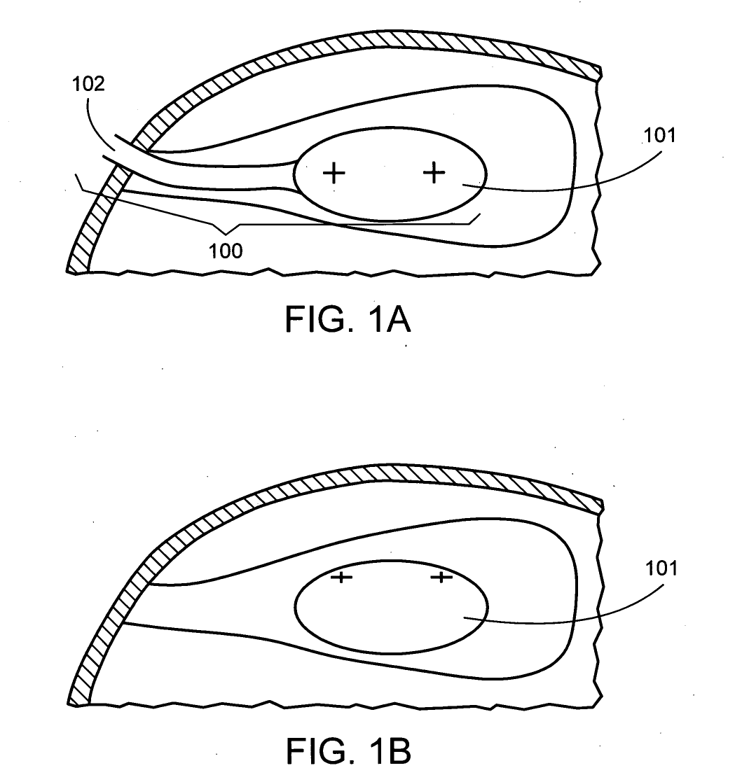

FIG. 1 depicts a lack of rotational stability often encountered with conventional expandable members. In FIG. 1A, a medical device 100 of the prior art comprising an expandable member 101 is delivered into a cavity within a patient through an incision in the patient's skin using a delivery means 102. For example, a conventional medical balloon containing a radiation-emitting source is inserted into a patient, following removal of a cancerous tumor, using a catheter so that the balloon is initially located adjacent to the tissue from which the tumor was removed. In that initial configuration, the balloon will be positioned so that it emits radiation to the targeted tissue. As seen in FIG. 1B, over time the expandable member 101 rotates upward within the cavity, away from the viewing angle. Rotation typically occurs with conventional medical balloons due to the accumulation of bodily fluids, such as blood, heme, etc., within the cavity. For example, blood may accumulate at a lumpectomy site following surgery. This accumulation effectually causes the balloon to float and/or shift around within the cavity. As a result, the radiation is misdirected to healthy tissue, having potentially detrimental effects.

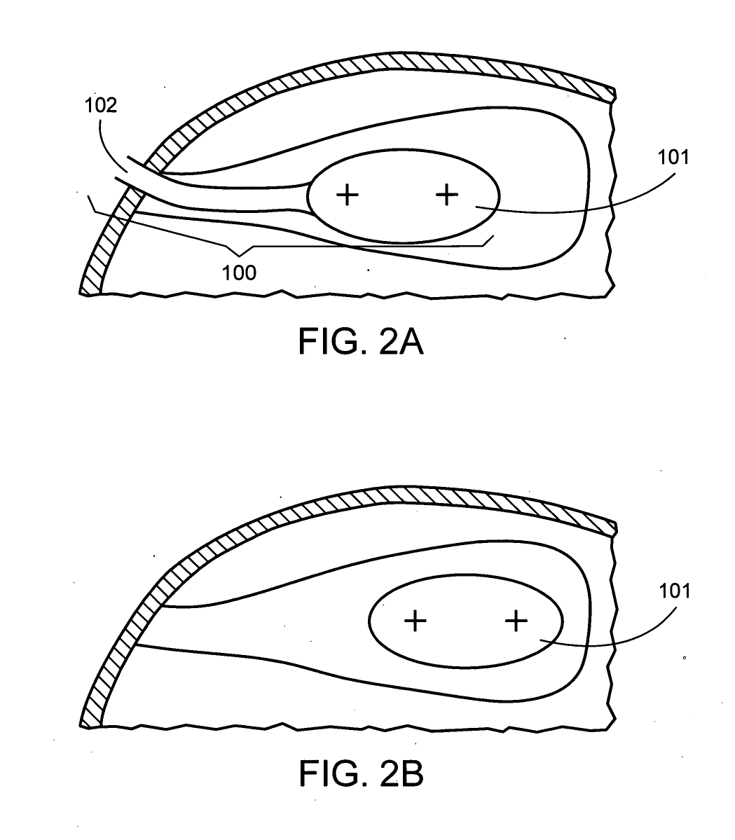

FIG. 2 depicts a lack of longitudinal stability often encountered with conventional expandable members. In FIG. 2A, a medical device of the prior art 100 comprising an expandable member 101 is delivered into a cavity within a patient through an incision in the patient's skin using a delivery means 102. For example, a conventional medical balloon containing a radiation-emitting source is inserted into a patient, following removal of a cancerous tumor, using a catheter so that the balloon is initially located adjacent to the tissue from which the tumor was removed. In that initial configuration, the balloon will be positioned so that it emits radiation to the targeted tissue. As seen in FIG. 2B, over time the expandable member 101 moves longitudinally within the cavity, to the right from the viewing angle. Longitudinal movement typically occurs with conventional medical balloons due to the accumulation of bodily fluids, such as blood, heme, etc., within the cavity. For example, blood may accumulate at a lumpectomy site following surgery. This accumulation effectually causes the balloon to float and/or shift around within the cavity. As a result, the radiation is misdirected to healthy tissue, having potentially detrimental effects.

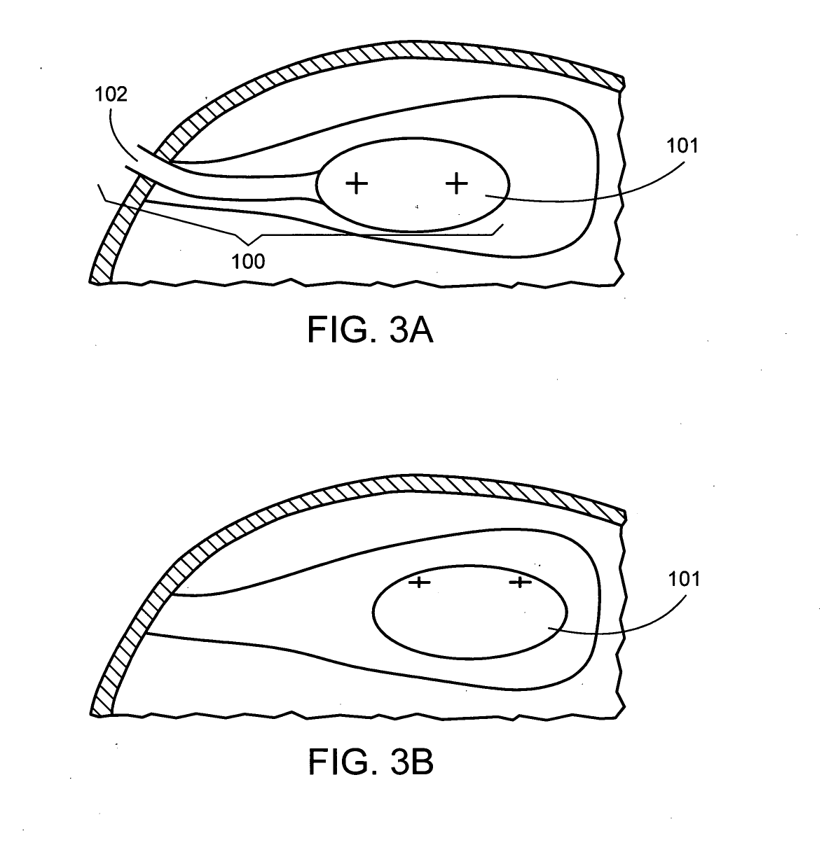

FIG. 3 depicts a lack of combined rotational and longitudinal stability often encountered with conventional expandable members. In FIG. 3A, a medical device 100 of the prior art comprising an expandable member 101 is delivered into a cavity within a patient through an incision in the patient's skin using a delivery means 102. For example, a conventional medical balloon containing a radiation-emitting source is inserted into a patient, following removal of a cancerous tumor, using a catheter so that the balloon is initially located adjacent to the tissue from which the tumor was removed. In that initial configuration, the balloon will be positioned so that it emits radiation to the targeted tissue. As seen in FIG. 3B, over time the expandable member 101 rotates upward and moves longitudinally within the cavity, to the right from the viewing angle. As a result, the radiation is misdirected to healthy tissue, having potentially detrimental effects.

As used herein, the term “fixation element” includes any physical configuration that achieves the improved rotational and/or longitudinal stability, including, for example, wing, fin, spike, and barb configurations. The fixation elements curb rotation by minimizing movement after placement. The fixation elements enhance longitudinal stability by providing greater structural support. In turn, this support minimizes situations such as inconsistent shaping encountered with conventional expandable members.

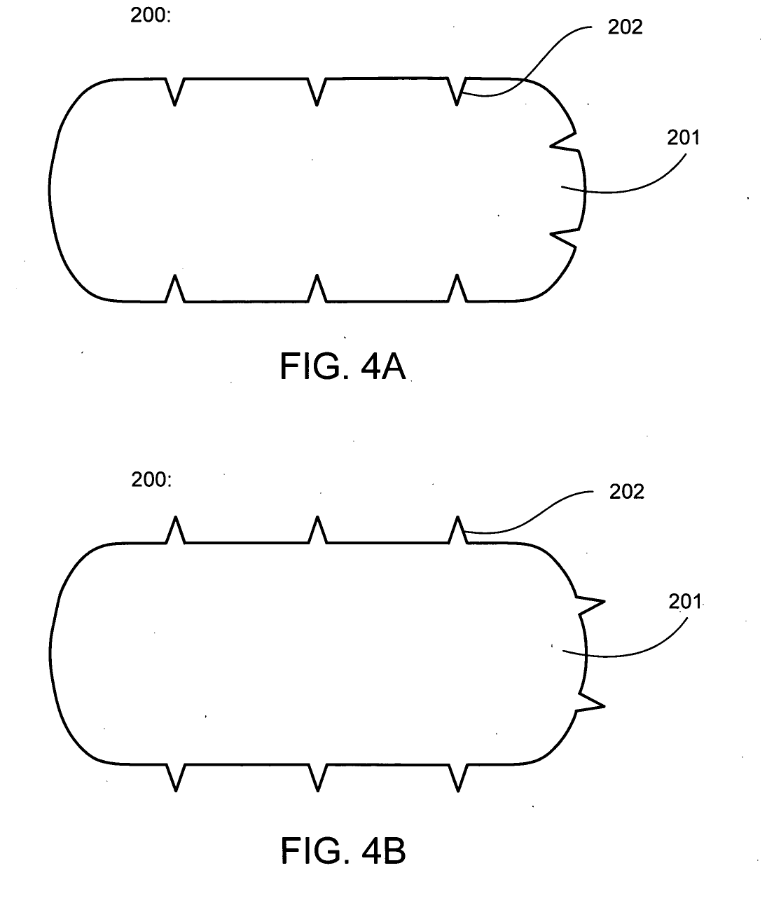

FIG. 4 depicts an embodiment of the invention. In FIG. 4A, a medical device 200 in accordance with an embodiment of the invention, comprising an expandable member 201 and a plurality of fixation elements 202 (spikes) recessed within the member, is depicted prior to expansion. For example, the expandable member 201 may be a balloon, having dimensions of approximately 6 centimeters in diameter, made of polyurethane and/or silicone whose fixation elements 202 are made of the same material and are integrated in the construction of the expandable member 201. The expandable member 201 may be inflated with saline, air, or other suitable medium once the expandable member 201 is positioned at the desired therapy site. FIG. 4B depicts a medical device 200 comprising an expandable member 201 following expansion, whose external fixation elements 202 (spikes) now protrude from the expandable member 201 to the therapy site. The fixation elements 202 (spikes) need not protrude deeply into the tissue. Protrusion of the fixation elements 202 by, for example, from approximately 0 millimeters to approximately 3 millimeters is sufficient.

While in no way limiting, the expandable members of the invention may comprise biocompatible, radiation-resistant polymers, such as Silastic rubbers, polyurethanes, polyethylene, polypropylene, polyester, PVC, and C-Flex.

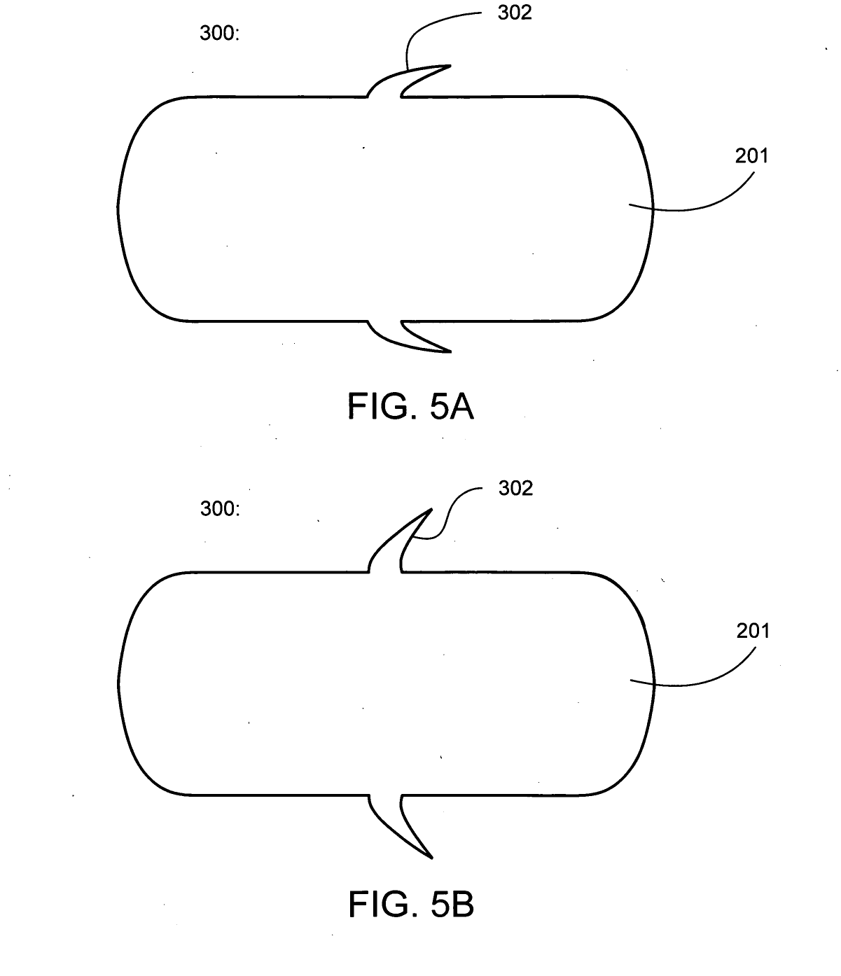

FIG. 5 depicts an embodiment of the invention. In FIG. 5A, a medical device 300 in accordance with an embodiment of the invention, comprising an expandable member 201 with a plurality of external fixation elements 302 (spikes) nesting proximal to the member 201, is depicted prior to expansion. For example, the expandable member 201 may be a balloon, having dimensions of approximately 6 centimeters in diameter, made of polyurethane and/or silicone whose fixation elements 302 are made of the same material and are integrated in the construction of the expandable member 201. The expandable member 201 may be inflated with saline, air, or other suitable medium once the expandable member 201 is positioned at the desired therapy site. FIG. 5B depicts the expandable member 201 following expansion, whose external fixation elements 302 (spikes) now protrude from, as opposed to nest immediately proximal to, the expandable member 201 to the therapy site. The fixation elements 302 (spikes) need not protrude deeply into the tissue. Protrusion of the fixation elements 302 by, for example, from approximately 0 millimeters to approximately 3 millimeters is sufficient.

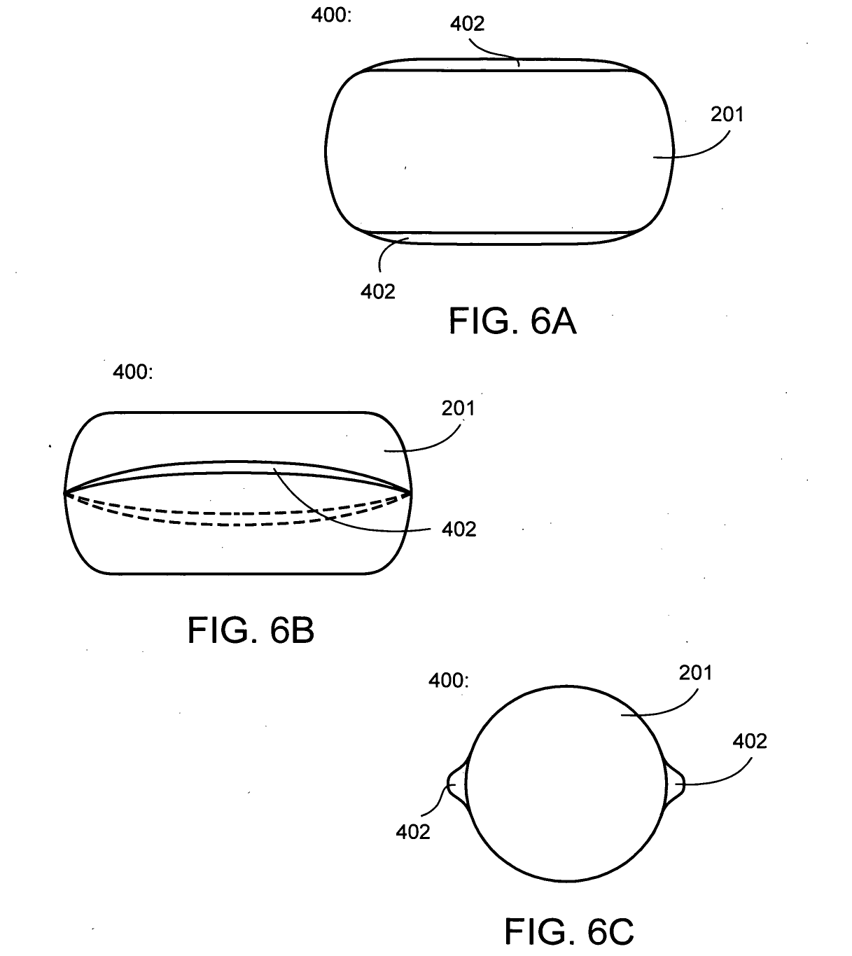

FIG. 6 depicts an embodiment of the invention. In FIG. 6A, a medical device 400 in accordance with an embodiment of the invention, comprising an expandable member 201 and a plurality of external fixation elements 402 (wings), is viewed from the side. Fixation elements such as wings enhance longitudinal stability by providing greater structural support. In turn, this support minimizes situations such as inconsistent shaping encountered with conventional expandable members. For example, the expandable member 201 may be a balloon, having dimensions of approximately 6 centimeters in diameter, made of polyurethane and/or silicone whose fixation elements 402 (wings) are made of the same material and are integrated in the construction of the expandable member 201. The expandable member 201 may be inflated with saline, air, or other suitable medium once the expandable member 201 is positioned at the desired therapy site. In FIG. 6B, an expandable member 201 and its fixation elements 402 (wings) are viewed from the top. In FIG. 6C, an expandable member 201 and its fixation elements 402 (wings) are viewed from an end.

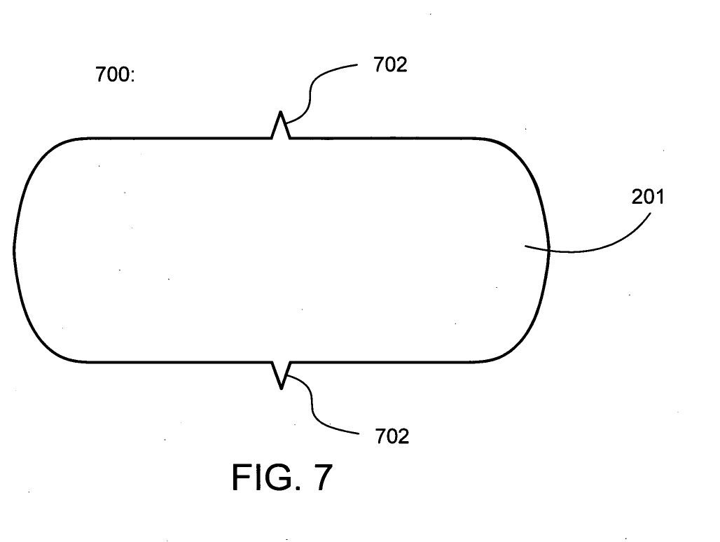

FIG. 7 depicts an embodiment of the invention. In FIG. 7, a medical device 700 in accordance with an embodiment of the invention, comprising an expandable member 201 and a plurality of fixation elements 702 (spikes) affixed to the outer surface of the expandable member, is viewed from the side. For example, the expandable member 201 may be a balloon, having dimensions of approximately 6 centimeters in diameter, made of polyurethane and/or silicone whose fixation elements 702 are made of the same material and are integrated in the construction of the expandable member 201. The expandable member 201 may be inflated with saline, air, or other suitable medium once the expandable member 201 is positioned at the desired therapy site. The fixation elements 702 (spikes) need not protrude deeply into the tissue. Protrusion of the fixation elements 702 by, for example, from approximately 0 millimeters to approximately 3 millimeters is sufficient. All that is needed is for the external fixation elements 702 to contact tissue within the patient to provide rotational and/or longitudinal stability of the expandable member within the patient.

In any embodiment, the number of external fixation elements will depend on the specific application and device dimensions. By way of example, the embodiments have from one up to six external fixation elements. Further by way of example, the fixation elements may be positioned in a manner equidistant from one another, such as every 60° around the diameter of the expandable member.

As used herein, the term “brachytherapy” refers to radiation therapy delivered by a spatially-confined source of therapeutic radiation. Often, the therapeutic radiation is administered within a patient's body, often at or near a tumor or other proliferative tissue disease site. Brachytherapy devices treat proliferative tissue disorders, such as cancerous tumors, by delivering radiation to the target area which contains both cancerous cells and healthy tissue. The radiation destroys the more radiosensitive cells, e.g., cancer cells, while hopefully minimizing damage to the surrounding healthy tissue. The most effective treatment delivers a dose above a minimum radiation dose necessary to destroy the proliferative tissue and below a maximum radiation dose to limit damage to healthy tissue. In addition to delivering a radiation dose within the proper range, brachytherapy devices may also deliver the radiation in a desired pattern. For example, it may be desirable to deliver radiation in a uniform three dimensional profile.

In use, the desired radiation dose is calculated based on factors such as the position of the radiation source, the type of radiation used, and the characteristics of the tissue and brachytherapy device. The brachytherapy device is then positioned within a tissue cavity and the dose is delivered. Unfortunately, variations in the brachytherapy device, in the surrounding tissue, or in the positioning of the radiation source can effect the delivered dose.

Some conventional brachytherapy devices include a catheter body member having a proximal end and a distal end, an inner spatial volume disposed proximate to the distal end of the catheter body member, an outer spatial volume defined by an expandable surface element disposed proximate to the distal end of the body member in a surrounding relation to the inner spatial volume, and a radiation source disposed in the inner spatial volume. The inner and outer spatial volumes are configured to provide an absorbed dose within a predetermined range throughout a target tissue. The target tissue is located between the outer spatial volume expandable surface and a minimum distance outward from the outer spatial volume expandable surface. The predetermined dose range is defined as being between a minimum prescribed absorbed dose for delivering therapeutic effects to tissue that may include cancer cells, and a maximum prescribed absorbed dose above which healthy tissue necrosis may result.

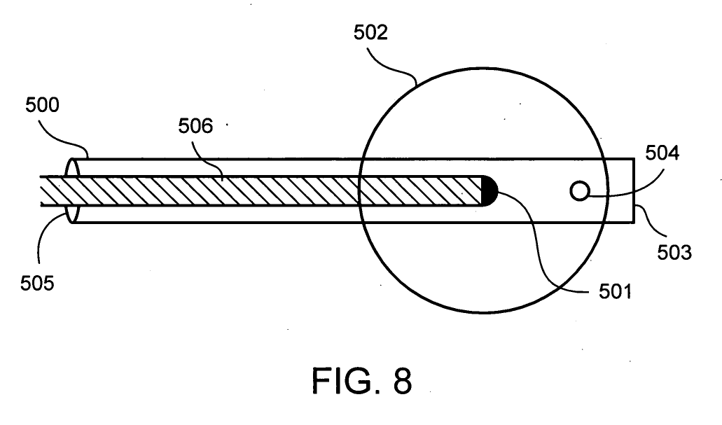

In other conventional brachytherapy devices of the prior art, such as the one depicted in FIG. 8, the catheter body member 500 may have a solid spherical radiation emitting material 501 within a spatial volume 502. The device has a distal end 503, an inflation port 504, and a proximal end 505. For example, radioactive micro spheres of the type available from the 3M Company of St. Paul, Minn., may be used. This radioactive source is loaded into the device after it has been implanted into the space formerly occupied by the excised tumor. For example, the solid radiation emitting material 501 is inserted through catheter 500 on a wire 506, using an afterloader. Such a solid radioactive core configuration offers an advantage in that it allows a wider range of radionuclides than if one is limited to liquids. Solid radionuclides that could be used with such a delivery device are currently generally available as brachytherapy radiation sources. However, such an apparatus can experience detrimental rotational and/or longitudinal instability.

Considering that brachytherapy seeks to deliver a predetermined radiation dosing profile solely to target tissue so that target tissue is treated and healthy tissue is not damaged, rotational and longitudinal stability is critical to safe and effective therapy. If the radiation source is not centered within, for example, the medical balloon, a predetermined asymmetric radiation dosing profile may be employed to protect sensitive tissues, such as skin and the chest wall. Alternatively, therapy may be designed to deliver a non-uniform dose of radiation, due to offset of, for example, the medical balloon within the cavity. In either case, movement of the balloon after placement may result in the target tissue receiving too little radiation and healthy, non-target tissue receiving deleterious radiation. The expandable members of the present invention curb and/or prevent undesirable post-placement movement and help maintain integrity of treatment planning profiles by preventing the need for recalculation and/or need to reposition the balloon, which can be painful to the patient and which can increase the risk of infection. Preventing post-placement movement is especially important where the radiation source is purposefully positioned to create an asymmetric radiation dosing profile.

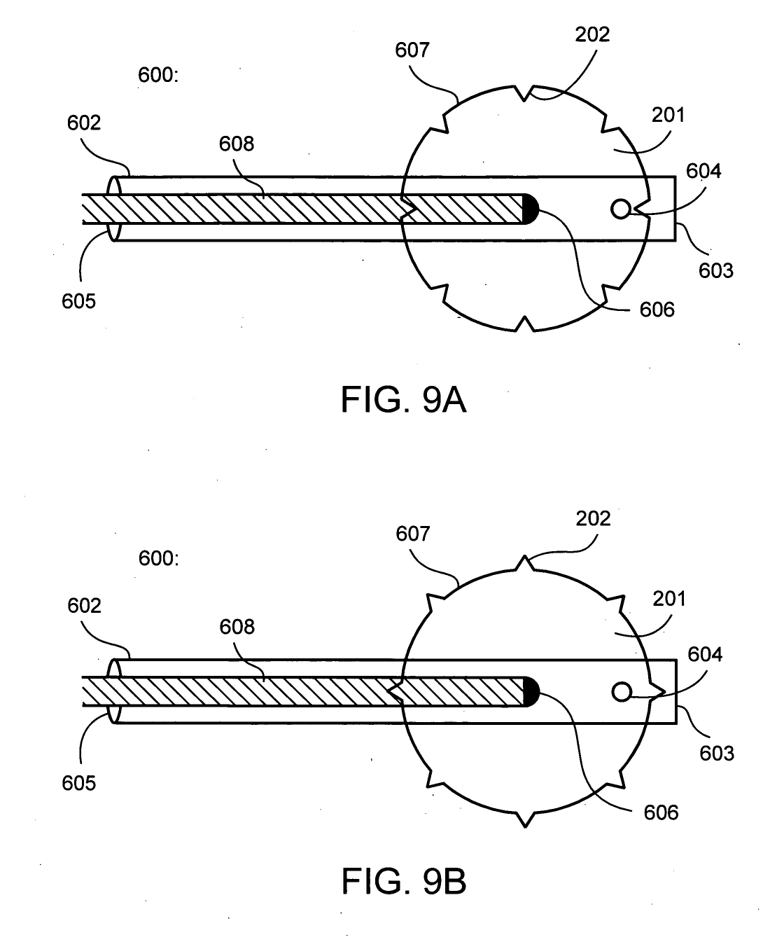

FIG. 9 depicts a brachytherapy apparatus 600 according to an embodiment of the present invention for delivering radioactive emissions to mammalian tissue wherein a radiation emitting source is contained within a spatial volume. In FIG. 9A, the brachytherapy apparatus 600 has an expandable member 201 with a plurality of external fixation elements 202 (spikes) recessed within the member, depicted prior to expansion. The brachytherapy apparatus 600 comprises a catheter 602, comprising a distal end 603, an inflation port 604, and a proximal end 605. The apparatus further comprises a radiation emitting material 606, which may be inserted through catheter 602 on a wire 608, within a spatial volume 607. The spatial volume 607 is defined by an expandable member 201 comprising an outer surface and a plurality of external fixation elements 202 (spikes) recessed within the member 201 prior to expansion. In FIG. 9B, the brachytherapy apparatus of FIG. 9A is depicted following expansion, using the inflation port 604, of the expandable member 201.

The catheter 602 of the brachytherapy apparatus 600 depicted in FIG. 9 provides a means for positioning the expandable member 201 within a tissue cavity and presents a path for delivering radiation emitting material and inflation material, if used. Although the exemplary catheter depicted in FIG. 9 has a tubular construction, one of skill in the art readily appreciates that the catheter 602 may have a variety of shapes and sizes. Catheters suitable for use in the invention include catheters which are known in the art. Although catheters may be constructed from a variety of materials, in one embodiment the catheter material is silicone, for example a silicone that is at least partially radio-opaque, thus facilitating x-ray localization of catheter after insertion. Catheters may also include conventional adapters for attachment to a treatment fluid receptacle and the balloon, as well as devices, e.g., right-angle devices, for conforming the catheter to contours of the patient's body.

An advantage of the brachytherapy apparatus of the present invention is that it provides for treatment of tissue surrounding a cavity left by surgical removal of a tumor in a living patient. Because the expandable members of the brachytherapy apparatus of the present invention may by intraoperatively placed in the cavity formerly occupied by the tumor, a means for subsequent treatment of any residual tumor and/or infiltrating tumor cells is provided, without having to make additional surgical incisions. Yet another advantage of the expandable members of the present invention is their natural compliance to conform to the outline of the cavity to be treated, allowing for close approximation of the member to the treatment site.

The brachytherapy apparatus of the invention can be used in the treatment of a variety of malignant tumors, and is especially useful for in the treatment of brain and breast tumors.

Many breast cancer patients are candidates for breast conservation surgery, also known as lumpectomy, a procedure that is generally performed on early stage, smaller tumors. Breast conservation surgery is typically followed by postoperative radiation therapy. Studies report that 80% of breast cancer recurrences after conservation surgery occur near the original tumor site, strongly suggesting that a tumor bed “boost” of local radiation to administer a strong direct dose may be effective in killing any remaining cancer and preventing recurrence at the original site. The apparatus described herein can be used for either the primary or boost therapy. Numerous studies and clinical trials have established equivalence of survival for appropriate patients treated with conservation surgery plus radiation therapy compared to mastectomy.

Surgery and radiation therapy are also the standard treatments for malignant solid brain tumors. The goal of surgery is to remove as much of the tumor as possible without damaging vital brain tissue. The ability to remove the entire malignant tumor is limited by its tendency to infiltrate adjacent normal tissue. Partial removal reduces the amount of tumor to be treated by radiation therapy and, under some circumstances, helps to relieve symptoms by reducing pressure on the brain.

A method according to the invention for treating these and other malignancies begins by surgical resection of a tumor site to remove at least a portion of the cancerous tumor and create a resection cavity. Following tumor resection, but prior to closing the surgical site, the surgeon intra-operatively places a brachytherapy apparatus comprising an expandable member and at least one external fixation element as described herein, but without having the radioactive source material loaded, into the tumor resection cavity. Once the patient has sufficiently recovered from the surgery, the brachytherapy apparatus is loaded with a radiation emitting source. The radioactive source dwells in the catheter until the prescribed dose of radiotherapy is delivered, typically for approximately a week or less. The radiation source is then retrieved and the catheter is removed. The radiation treatment may end upon removal of the brachytherapy apparatus, or the brachytherapy may be supplemented by further doses of radiation supplied externally.

Radiation emitting sources useful for the present invention include any radiation source which can deliver radiation to treat proliferative disorders, including high-dose radiation, medium-dose radiation, low-dose radiation, pulsed-dose radiation, external beam radiation, and combinations thereof. Such sources include predetermined radionuclides, for example, I-125, I-131, Yb-169, as well as other sources of radiation, such as radionuclides that emit photons, beta particles, or other therapeutic rays. Radiation emitting sources useful for the present invention may operate alone, or may be used in conjunction with radioactive ray absorbent material, such as air, water, and/or contrast materials. Radiation emitting sources useful for the present invention may include a single solid sphere, or may comprise a plurality of radioactive particles strategically placed so as to radiate in one or more directions with equal or varying intensities.

The radiation emitting source may also be a radioactive fluid made from any solution of radionuclide(s). Such a radioactive fluid may also be produced using a slurry of suitable fluid containing small particles of solid radionuclides, such as Au-198 and Y-90. Radionuclides may also be embodied in a gel.

By employing an expandable member with at least one external fixation element in the methods of the present invention, rotational and/or longitudinal stability is greatly increased, thereby resulting in more precise therapy. Similarly, brachytherapy apparatus of the present invention employing an expandable member of the present invention with increased rotational and/or longitudinal stability offers brachytherapy with greater precision.

A person skilled in the art will appreciate the foregoing as only illustrative of the principles of the invention, and that various modifications can be made by those skilled in the art without departing from the scope and spirit of the invention.

What is claimed is:

1. A brachytherapy apparatus for delivering radioactive emissions to a patient, comprising:
(a) an expandable member for placement within a patient comprising an outer surface and at least one external fixation element affixed to said outer surface;
(b) a catheter comprising a proximal end, a distal end, and spatial volume at said distal end, wherein said spatial volume is defined by said expandable member; and
(c) a radiation source position disposed in said spatial volume,
wherein said external fixation element contacts tissue within said patient to provide stability to said expandable member within said patient.

2. The brachytherapy apparatus of claim 1, wherein said at least one external fixation element is selected from the group consisting of wings, fins, spikes, and barbs.

3. A brachytherapy apparatus for delivering radioactive emissions to a patient, comprising:
(a) an expandable member for placement within a patient comprising an outer surface and at least one external fixation element, wherein upon expansion of said expandable member, said external fixation element projects from said outer surface to contact tissue within said patient;
(b) a catheter comprising a proximal end, a distal end, and spatial volume at said distal end, wherein said spatial volume is defined by said expandable member; and
(c) a radiation source position disposed in said spatial volume,
wherein said contact between said external fixation element and said patient tissue provides stability to said expandable member within said patient.

4. The brachytherapy apparatus of claim 3, wherein said at least one external fixation element is selected from the group consisting of wings, fins, spikes, and barbs.

5. The brachytherapy apparatus of claim 3, wherein said at least one external fixation element, prior to expansion of said member, is recessed within said member.

6. The brachytherapy apparatus of claim 3, wherein said at least one external fixation element, prior to expansion of said member, is nesting proximal to said member.

7. A method for performing a brachytherapy procedure in a patient, comprising:
inserting into said patient a catheter comprising a proximal end, a distal end, and spatial volume at said distal end, wherein said spatial volume is defined by an expandable member comprising an outer surface and at least one external fixation element on said outer surface;
inflating or expanding said expandable member to a volume sufficient to cause said at least one external fixation element to contact tissue within said patient;
inserting a radiation source in the spatial volume of said catheter; and
removing said radiation source and said catheter from said patient,
wherein said contact is sufficient to hinder movement of said expandable member within said tissue.

8. The method for performing brachytherapy of claim 7, wherein upon expansion of said expandable member said at least one external fixation element projects from said outer surface to contact said tissue.

9. The method for performing brachytherapy of claim 7, wherein said at least one external fixation element is affixed to said outer surface of said expandable member.

10. The method for performing brachytherapy of claim 7, wherein said at least one external fixation element is selected from the group consisting of wings, fins, spikes, and barbs.

11. The method for performing brachytherapy of claim 7, wherein upon expansion of said expandable member said at least one external fixation element projects from said outer surface to contact said tissue and further wherein said at least one external fixation element, prior to expansion of said member, is recessed within the member.

12. The method for performing brachytherapy of claim 7, wherein upon expansion of said expandable member said at least one external fixation element projects from said outer surface to contact said tissue and further wherein said at least one external fixation element, prior to expansion of said member, is nesting proximal to said member.
